(19) United States
(12) Patent Application Publication
(10) Pub. No.: US 2025/0269235 A1
(43) Pub. Date: Aug. 28, 2025

(54) ELECTRONIC DEVICE AND METHOD FOR DETECTING USER'S EXERCISE

(71) Applicant: SAMSUNG ELECTRONICS CO., LTD., Suwon-si (KR)

(72) Inventors: Changlim CHOI, Suwon-si (KR); Jeongwan KANG, Suwon-si (KR); Chaeheun LEE, Suwon-si (KR); Hoseong LEE, Suwon-si (KR)

(73) Assignee: SAMSUNG ELECTRONICS CO., LTD., Suwon-si (KR)

(21) Appl. No.: 19/208,015

(22) Filed: May 14, 2025

Related U.S. Application Data
(63) Continuation of application No. PCT/KR2023/018253, filed on Nov. 14, 2023.

(30) Foreign Application Priority Data
Nov. 14, 2022 (KR) 10-2022-0151886
Dec. 12, 2022 (KR) 10-2022-0172531

(51) Int. Cl. A63B 24/00; A63B 71/06; G04G 21/02

(52) U.S. Cl. CPC A63B 24/0062; A63B 71/0622; A63B 2024/0068; A63B 2220/30; A63B 2220/40; A63B 2220/56; A63B 2220/803; A63B 2225/20; A63B 2230/065; G04G 21/025

(57) ABSTRACT

An electronic device includes an inertia sensor, a global navigation satellite system (GNSS) sensor, a heart rate (HR) sensor, a memory including one or more storage media and storing instructions, a display, and a processor, and the instructions, when executed by the at least one processor individually or collectively, cause the electronic device to: identify a specific exercise based on a signal detected through the inertia sensor; determine whether the GNSS sensor is available; identify a movement speed of the electronic device based on the GNSS sensor; and based on determining that the identified movement speed is within a designated speed range, determine that a user is performing the specific exercise and execute a function of the electronic device.

### CROSS-REFERENCE TO RELATED APPLICATIONS

This application is a continuation of International Application No. PCT/KR2023/018253, filed on Nov. 14, 2023, which is based on and claims priority to Korean Patent Application No. 10-2022-0151886, filed on Nov. 14, 2022, in the Korean Intellectual Property Office, and Korean Patent Application No. 10-2022-0172531, filed on Dec. 12, 2022, in the Korean Intellectual Property Office, the disclosures of which are incorporated by reference herein in their entireties.

### BACKGROUND

### 1. Field

The disclosure relates to an electronic device and a method for detecting a user's exercise.

### 2. Description of Related Art

Recently, electronic devices including sensors capable of measuring user's biometric information and/or fitness data have been developed.

The electronic devices may typically be carried in a pocket or hand for use while the user moves, and may also have a form of being wearable on a part of the human body or various structures. The electronic device may offer health and fitness information services by utilizing an ability thereof to be worn on the user's body.

Since users often start exercising without remembering to record their exercise using an electronic device, the electronic device may output a notification requesting to record an exercise when it is determined that the user is performing a specific exercise.

The above-described information may be provided as related art for the purpose of assisting in understanding the disclosure. No assertion or decision is made as to whether any of the above might be applicable as prior art with regard to the disclosure.

An electronic device may detect a specific exercise, based on a signal pattern detected through a sensor.

However, detecting a specific exercise based on one sensor may require long time for monitoring a signal pattern to improve accuracy of detection of the specific exercise by the electronic device. Physical activities of a user with similar signal patterns make it difficult to accurately recognize specific exercises. And so, although a user of such sensor might themselves might themselves recognize a start of their exercise, the sensor and device technology using such sensor is deficient in that it cannot simply be commanded to detect a start of exercise without first accounting for those above deficiencies in the technology itself.

Furthermore, in case that the user performs an exercise (e.g., cycling) of which notification is difficult to be identified, the electronic device may have a problem of deleting an exercise record before the notification is identified.

### SUMMARY

Accordingly to an aspect of the disclosure, an electronic device includes: an inertia sensor; a global navigation satellite system (GNSS) sensor; a heart rate (HR) sensor; a memory, including one or more storage media, storing instructions; a display; and at least one processor including processing circuitry, wherein the instructions, when executed by the at least one processor individually or collectively, cause the electronic device to: identify a specific exercise by differentiating the specific exercise from other exercises, based on a signal detected through the inertia sensor; determine whether the GNSS sensor is available; identify a movement speed of the electronic device based on the GNSS sensor; based on determining that the identified movement speed is greater than or equal to a first specific speed, determine whether the identified movement speed is greater than a second specific speed; and based on determining that the identified movement speed is less than the second specific speed, determine whether the identified movement speed is within a designated speed range.

The instructions, when executed by the at least one processor individually or collectively, may cause the electronic device to: based on determining that the identified movement speed is not within the designated speed range, identify a heart rate of the user by using the HR sensor; based on determining that the identified heart rate exceeds a specific heart rate and exceeds a designated heart rate range, determine that the user is performing the specific exercise and execute the function; and based on determining that the identified heart rate exceeds the specific heart rate and is within the designated heart rate range, monitor the heart rate of the user by using the HR sensor.

The instructions, when executed by the at least one processor individually or collectively, may cause the electronic device to: based on determining that the identified heart rate exceeds the specific heart rate and is within the designated heart rate range, monitor the heart rate of the user by using the HR sensor.

The designated speed range may be greater than the first specific speed and is less than the second specific speed.

The instructions, when executed by the at least one processor individually or collectively, may cause the electronic device to: based on determining that the GNSS sensor is not available, identify the heart rate of the user by using the HR sensor; and based on determining that the identified heart rate exceeds a specific heart rate and exceeds the designated heart rate range, determine that the user is performing the specific exercise and execute the function.

The instructions, when executed by the at least one processor individually or collectively, may cause the electronic device to: determine the specific exercise based on a signal acquired based on at least one of the inertia sensor, the GNSS sensor, and the HR sensor; perform a recording of the inertia sensor and the specific exercise before a notification of the inertia sensor; and display a notification for the specific exercise on the display, and perform a countdown for starting recording for the specific exercise.

The instructions, when executed by the at least one processor individually or collectively, may cause the electronic device to: based on determining that the countdown is expired or a user's identification being identified, perform recording of the specific exercise after the notification; and aggregate and store a first record of the specific exercise before the notification is displayed and a second record for the specific exercise after the notification, and display an aggregated record of an aggregation of the stored first record and the stored second record.

The instructions, when executed by the at least one processor individually or collectively, may cause the electronic device to: determine a slope based on a pressure change while determining whether the user is performing the specific exercise.

The instructions, when executed by the at least one processor individually or collectively, may cause the electronic device to: determine a movement distance based on a factor according to the slope and a speed for each of a plurality of sections from the recording; determine a heart rate change of the user while determining whether the user is performing the specific exercise; and determine the movement distance based on a factor according to the heart rate change of the user and the speed for each of the plurality of sections.

According to an aspect of the disclosure, a method of detecting an exercise of a user of an electronic device, includes: identifying a specific exercise by differentiating the specific exercise from other exercises, based on a signal detected through an inertia sensor; determining whether a global navigation satellite system (GNSS) sensor of the electronic device is available; identifying a movement speed of the electronic device, based on the GNSS sensor; based on determining that the identified movement speed is greater than or equal to a first specific speed, determining whether the identified movement speed is greater than a second specific speed; based on determining that the identified movement speed is less than the second specific speed, determining whether the identified movement speed is within a designated speed range; and based on determining that the identified movement speed is within the designated speed range, determining that the user is performing the specific exercise and executing a function of the electronic device.

The method may further include: based on determining that the identified movement speed is not within the designated speed range, identifying a heart rate of the user by using a heart rate (HR) sensor; based on determining that the identified heart rate exceeds a specific heart rate and exceeds a designated heart rate range, determining that the user is performing the specific exercise and executing a function.

The method may further include: based on determining that the identified heart rate exceeds the specific heart rate and is within the designated heart rate range, monitoring the heart rate of the user by using the HR sensor.

The designated speed range may be greater than the first specific speed and is less than the second specific speed.

The method may further include: based on determining that the GNSS sensor is not available, identifying the heart rate of the user by using the HR sensor; and based on determining that the identified heart rate exceeds a specific heart rate and exceeds the designated heart rate range, determining that the user is performing the specific exercise and executing the function.

The method may further include: determining the specific exercise based on a signal acquired based on at least one of the inertia sensor, the GNSS sensor, and the HR sensor; performing a recording of the inertia sensor and the specific exercise before a notification of the inertia sensor; and displaying a notification for the specific exercise on a display of the electronic device, and performing a countdown for starting recording for the specific exercise.

The method may further include: based on determining that the countdown is expired or that a user's identification being identified, performing recording of the specific exercise after the notification.

The method may further include: aggregating and storing a first record of the specific exercise before the notification is displayed and a second record for the specific exercise after the notification and displaying an aggregated record of an aggregation of the stored first record and the stored second record.

The method may further include: determining a slope based on a pressure change while determining whether the user is performing the specific exercise; and determining a movement distance based on a factor according to the slope and a speed for each of a plurality of sections from the recording.

The method may further include: determining a heart rate change of the user while determining whether the user is performing the specific exercise; and determining a movement distance based on a factor according to the heart rate change of the user and the speed for each of a plurality of sections of the recording.

According to one or more embodiments of the disclosure, the electronic device and the method for detecting user's exercise may detect a physical activity of the user by using various sensors so as to accurately detect a specific exercise in a short period of time.

According to one or more embodiments of the disclosure, the electronic device and the method for detecting an exercise of a user may compensate for an exercise record before a notification of a specific exercise occurs in the total exercise record so as to provide an accurate exercise record to the user.

According to one or more embodiments of the disclosure, the electronic device and the method for detecting an exercise of a user may provide, to the user, an interface corresponding to a specific exercise when a notification with respect to the specific exercise occurs so as to improve user safety.

### BRIEF DESCRIPTION OF THE DRAWINGS

The above and other aspects, features, and advantages of specific embodiments of the present disclosure will be more apparent from the following description with reference to the accompanying drawings, in which:

FIG. 1 is a block view illustrating an electronic device in a network environment according to various embodiments of the disclosure;

FIG. 2 is a flowchart illustrating a method for an electronic device to detect an exercise of a user according to one or more embodiments of the disclosure;

FIG. 3 is a flowchart illustrating a method for an electronic device to detect an exercise of a user according to one or more embodiments of the disclosure;

FIG. 4 is a flowchart illustrating an operation of FIG. 2 and an operation of FIG. 3 according to one or more embodiments of the disclosure;

FIG. 5A is a view illustrating a screen before an electronic device identifies a specific exercise according to one or more embodiments of the disclosure;

FIG. 5B illustrates a screen while an electronic device recognizes whether it is a specific exercise according to one or more embodiments of the disclosure;

FIG. 5C illustrates a screen where an electronic device determines that it is a specific exercise and displays a notification according to one or more embodiments of the disclosure;

FIG. 5D illustrates a screen where an electronic device performs recording for a user's specific exercise by using a GNSS sensor and an HR sensor according to one or more embodiments of the disclosure;

FIG. 6 is a flowchart illustrating a method for determining a movement distance in an operation of performing recording for a specific exercise before a notification in FIG. 4 according to one or more embodiments of the disclosure;

FIG. 7 is a graph illustrating a method for determining a movement distance in FIG. 6 according to one or more embodiments of the disclosure;

FIG. 8 is a flowchart illustrating a method for determining a movement distance in an operation of performing recording for a specific exercise before a notification in FIG. 4 according to one or more embodiments of the disclosure; and

FIG. 9 is a graph illustrating a method for determining a movement distance in FIG. 8 according to one or more embodiments of the disclosure.

### DETAILED DESCRIPTION

Hereinafter, embodiments of the disclosure will be described in detail with reference to the accompanying drawings so that those skilled in the art to which the disclosure pertains can easily implement the disclosure. However, the disclosure may be implemented in various forms and is not limited to embodiments set forth herein. With regard to the description of the drawings, the same or like reference signs may be used to designate the same or like elements. Also, in the drawings and the relevant descriptions, description of well-known functions and configurations may be omitted for the sake of clarity and brevity.

An electronic device may detect a specific exercise, based on a signal pattern detected through a sensor.

However, detecting a specific exercise based on one sensor may require relatively long time for monitoring a signal pattern to improve accuracy of detection of the specific exercise by the electronic device. Physical activities of a user with similar signal patterns may make it difficult to accurately recognize specific exercises.

Furthermore, in case that the user performs an exercise (e.g., cycling) of which notification is difficult to identified, the electronic device may delete an exercise record before the notification is identified.

The electronic device and the method for detecting an exercise of a user according to the disclosure may allow detection of an exercise performed by the user by using various sensors.

The electronic device and the method for detecting an exercise of a user according to the disclosure may compensate for an exercise record before a notification of a specific exercise occurs in the total exercise record.

The electronic device and the method for detecting an exercise of a user according to the disclosure may provide, to the user, an interface corresponding to a specific exercise when a notification with respect to the specific exercise occurs.

The electronic device and the method for detecting user's exercise according to the disclosure may detect a physical activity of the user by using various sensors so as to relatively accurately detect a specific exercise in a short period of time.

The electronic device and the method for detecting an exercise of a user according to the disclosure may compensate for an exercise record before a notification of a specific exercise occurs in the total exercise record so as to provide a relatively accurate exercise record to the user.

The electronic device and the method for detecting an exercise of a user according to the disclosure may provide, to the user, an interface corresponding to a specific exercise when a notification with respect to the specific exercise occurs so as to relatively improve user safety.

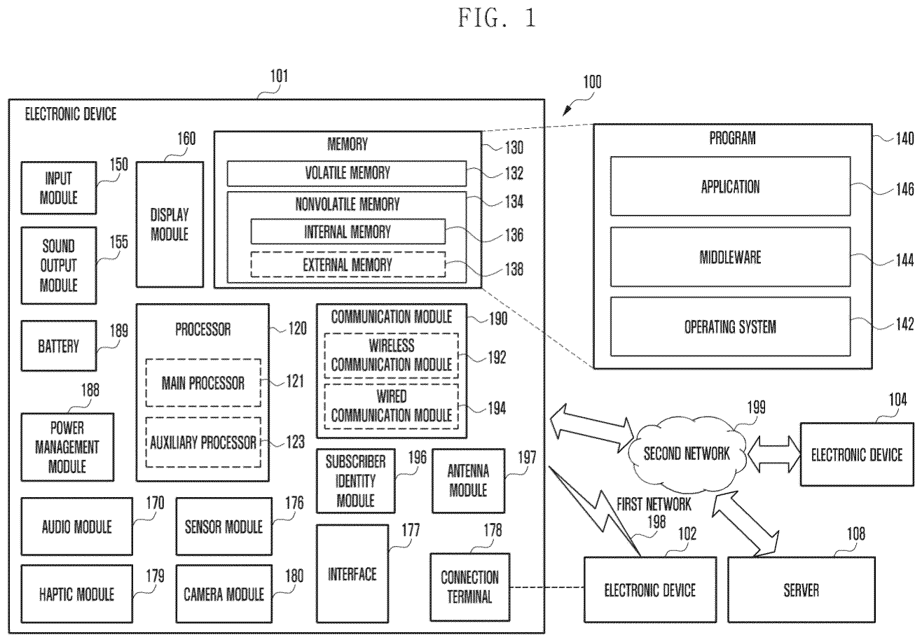

FIG. 1 is a block diagram illustrating an electronic device 101 in a network environment 100 according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IOT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

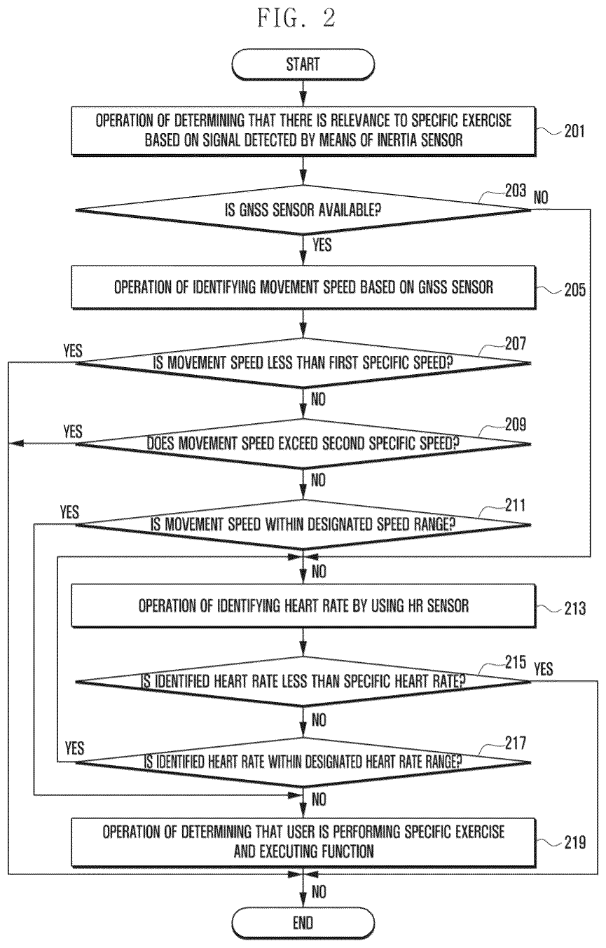

FIG. 2 is a flowchart illustrating a method for an electronic device 101 to detect an exercise of a user according to one or more embodiments of the disclosure.

In the following embodiment, respective operations may be sequentially performed, but are not necessarily sequentially performed. For example, the sequential position of each operation may be changed, or at least two operations may be performed in parallel.

According to one or more embodiments, it may be understood that operation 201, operation 202, operation 203, operation 204, operation 205, operation 206, operation 207, operation 208, operation 209, operation 210, operation 211, operation 212, operation 213, operation 214, operation 215, operation 216, operation 217, operation 218, and operation 219 are performed by a processor (e.g., the processor 120 in FIG. 1) of the electronic device (e.g., the electronic device 101 in FIG. 1).

In one or more embodiments of the disclosure, in operation 201, under control of the processor 120, the electronic device (e.g., the electronic device 101 in FIG. 1) may determine that there is relevance to a specific exercise based on a signal detected by means of an inertia sensor. The inertia sensor may detect inertia forces of motion to detect information related to the acceleration, velocity, direction, and distance of a moving object to be measured. For example, the inertia sensor may correspond to an acceleration sensor and/or a gyro sensor included in the sensor module (e.g., the sensor module 176 in FIG. 1).

In one or more embodiments of the disclosure, in operation 201, under control of the processor 120, the electronic device 101 may determine that there is relevance to a specific exercise in case that a signal detected through the inertia sensor has been detected for a predetermined time period (e.g., 2 minutes) or more.

For example, the signal detected through the inertia sensor may have a specific pattern. In case that a specific pattern persists and/or repeats for a specific time period, in operation 201, under control of the processor 120, the electronic device 101 may determine that it is a specific exercise.

For example, the specific exercise may be a cycling exercise.

In one or more embodiments of the disclosure, in operation 203, under control of the processor 120, the electronic device 101 may determine whether the GNSS sensor is available.

The GNSS sensor may be included in a communication module (e.g., the communication module 190 in FIG. 1). The GNSS sensor may include, for example, a global positioning system (GPS), a global navigation satellite system (GLONASS), Galileo, Beidou, a quasi-zenith satellite system (QZSS), and/or a navigation with Indian constellation (NAVIC).

The electronic device 101 configures the GNSS sensor to be in a standby state to reduce current consumption of the GNSS sensor. However, the electronic device 101 may, in case that a signal pattern associated with the specific exercise is detected through the inertia sensor, activate the GNSS sensor and identify whether it is the specific exercise according to the signal pattern.

In one or more embodiments of the disclosure, in operation 203, under control of the processor 120, the electronic device 101 may determine whether a GNSS signal is detected through the GNSS sensor.

In one or more embodiments of the disclosure, in operation 203, under control of the processor 120, the electronic device 101 may, in case that the GNSS signal is detected through the GNSS sensor, determine that the GNSS sensor is available and proceed to operation 205.

In one or more embodiments of the disclosure, in operation 203, under control of the processor 120, the electronic device 101 may, in case that no GNSS signal is detected through the GNSS sensor, determine that the GNSS sensor is unavailable and proceed to operation 213.

For example, a GNSS signal may be shaded when the electronic device 101 enters a tunnel or an interior of a building (e.g., a parking lot), and the GNSS signal may be difficult to be detected by a GNSS sensor.

In one or more embodiments of the disclosure, the electronic device 101 may, in case that the GNSS signal is detected through the GNSS sensor, determine whether the user is performing a specific exercise, by using the heart rate (HR) sensor, without information for a movement speed.

For example, when the user is performing a specific exercise, the heart rate may be greater than a designated heart rate.

In one or more embodiments of the disclosure, in operation 205, under control of the processor 120, the electronic device 101 may identify a movement speed of the electronic device 101 based on the GNSS sensor.

The electronic device 101 according to one or more embodiments of the disclosure is assumed to be worn or carried by the user. Accordingly, in operation 205, the operation in which the electronic device 101 identifies the movement speed of the electronic device based on the GNSS sensor may correspond to an operation of identifying the movement speed of the user.

In one or more embodiments of the disclosure, in operation 207, under control of the processor 120, the electronic device 101 may determine whether the movement speed identified based on the GNSS sensor is less than a first specific speed.

In one or more embodiments of the disclosure, in operation 207, under control of the processor 120, the electronic device 101 may determine whether the movement speed identified based on the GNSS sensor is slower than the first specific speed. For example, the first specific speed may be 6.9 km/h.

In one or more embodiments of the disclosure, in case that it is determined that the movement speed identified based on the GNSS sensor is slower than the first specific speed in operation 207, under control of the processor 120, the electronic device 101 may determine that the signal detected based on the inertia sensor of the electronic device 101 is a movement irrelevant to the specific exercise. For example, the movement having a movement speed slower than the first specific speed may correspond to a movement of pushing a stroller or cart.

In one or more embodiments of the disclosure, in operation 207, under control of the processor 120, the electronic device 101 may, in case that the movement speed identified based on the GNSS sensor is faster than or equal to the first specific speed, proceed to operation 209.

In one or more embodiments of the disclosure, in operation 207, under control of the processor 120, the electronic device 101 may, in case that the movement speed identified based on the GNSS sensor is faster than the first specific speed, proceed to operation 209.

In one or more embodiments of the disclosure, in operation 209, under control of the processor 120, the electronic device 101 may determine whether the movement speed identified based on the GNSS sensor exceeds a second specific speed.

In one or more embodiments of the disclosure, in operation 209, under control of the processor 120, the electronic device 101 may determine whether the movement speed identified based on the GNSS sensor is faster than the second specific speed. For example, the second specific speed may be 70.1 km/h.

In one or more embodiments of the disclosure, in operation 209, under control of the processor 120, the electronic device 101 may, in case that the movement speed identified based on the GNSS sensor is slower than or equal to the second specific speed, proceed to operation 211.

In one or more embodiments of the disclosure, in case that it is determined that the movement speed identified based on the GNSS sensor is faster than the second specific speed in operation 209, under control of the processor 120, the electronic device 101 may determine that the signal detected based on the inertia sensor of the electronic device 101 is a movement irrelevant to the specific exercise. For example, the exercise having a speed faster than the second specific speed may correspond to a vehicle movement.

In one or more embodiments of the disclosure, in operation 209, under control of the processor 120, the electronic device 101 may, in case that the movement speed identified based on the GNSS sensor is slower than the second specific speed, proceed to operation 211.

In one or more embodiments of the disclosure, in operation 211, under control of the processor 120, the electronic device 101 may determine whether the movement speed is within a designated speed range. For example, the designated speed range may be from 7 to 20 km/h.

In one or more embodiments of the disclosure, in operation 211, under control of the processor 120, the electronic device 101 may, in case that the movement speed is within the designated speed range, proceed to operation 219.

In one or more embodiments of the disclosure, in operation 211, under control of the processor 120, the electronic device 101 may, in case that the movement speed is not within the designated speed range, proceed to operation 213.

For example, the case where the movement speed is not within the designated speed range may be a case where the movement speed is within a designated speed range (e.g., from about 20.1 to about 70.1 km/h) faster than the first specific speed (e.g., 6.9 km/h), and slower than the second specific speed (e.g., 70.1 km/h), but is not within the designated speed range (e.g., 7 to 20 km/h). In case that the electronic device 101 is within the designated speed range (e.g., about 20.1 to about 70 km/h), the electronic device 101 may measure the heart rate by using a biometric sensor (e.g., the heart rate (HR) sensor) included in the sensor module 176.

In one or more embodiments of the disclosure, in operation 211, under control of the processor 120, the electronic device 101 may identify the heart rate by using the heart rate (HR) sensor.

In one or more embodiments of the disclosure, in operation 215, under control of the processor 120, the electronic device 101 may determine whether the identified heart rate of the user is less than a designated heart rate.

In one or more embodiments of the disclosure, in operation 215, under control of the processor 120, the electronic device 101 may determine whether the identified heart rate of the user is lower than the designated heart rate. For example, the designated heart rate may be 74.9 bpm (beats per minute).

In one or more embodiments of the disclosure, in operation 215, under control of the processor 120, the electronic device 101 may, in case that the identified heart rate of the user is less than the designated heart rate, determine that there is no relevance to the specific exercise.

In one or more embodiments of the disclosure, in case that it is determined that the identified heart rate of the user is greater than or equal to the designated heart rate in operation 215, under control of the processor 120, the electronic device 101 may proceed to operation 217.

In one or more embodiments of the disclosure, in case that it is determined that the identified heart rate of the user is higher than the designated heart rate in operation 215, under control of the processor 120, the electronic device 101 may proceed to operation 217.

In one or more embodiments of the disclosure, in operation 217, under control of the processor 120, the electronic device 101 may determine whether the identified heart rate is within a designated heart rate range. For example, the designated heart rate range may be from 75 bpm to 120 bpm.

In one or more embodiments of the disclosure, in case that the identified heart rate is within the designated heart rate range in operation 217, under control of the processor 120, the electronic device 101 may proceed to operation 213. In case that the identified heart rate is within the designated heart rate range, under control of the processor 120, the electronic device 101 may monitor the heart rate of the user again.

In one or more embodiments of the disclosure, in case that the identified heart rate is not within the designated heart rate range in operation 217, under control of the processor 120, the electronic device 101 may proceed to operation 219.

In one or more embodiments of the disclosure, in case that the identified heart rate exceeds the designated heart rate range in operation 217, under control of the processor 120, the electronic device 101 may proceed to operation 219.

In one or more embodiments of the disclosure, in operation 219, under control of the processor 120, the electronic device 101 may determine that the user is performing the specific exercise and execute a function. The function executed by the electronic device 101 is an operation of displaying a notification associated with the specific exercise on a user interface on the display module (e.g., the display module 160 in FIG. 1) including the display. The function executed by the electronic device 101 may store, in the memory (e.g., the memory 130 in FIG. 1), an exercise record before the notification is displayed on the user interface on the display module 160 including the display and an exercise record after the notification is displayed.

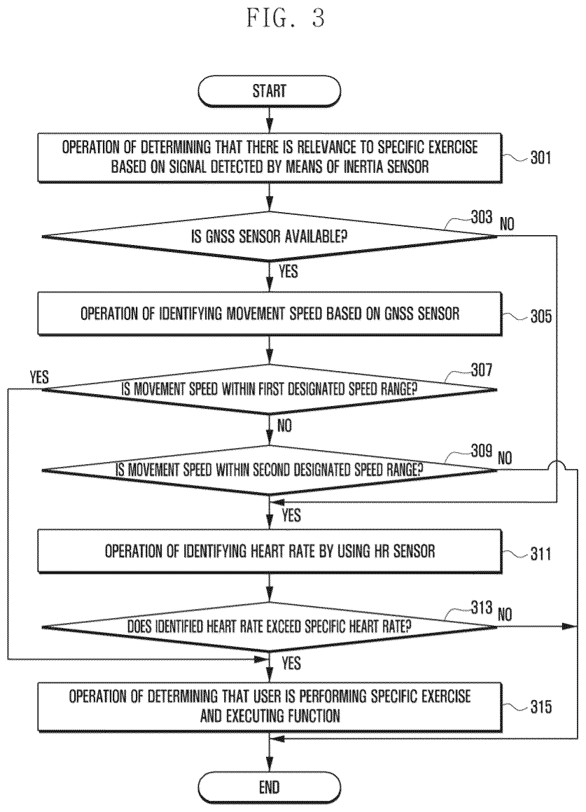

FIG. 3 is a flowchart illustrating a method for an electronic device 101 to detect an exercise of a user according to one or more embodiments of the disclosure.

In the following embodiment, respective operations may be sequentially performed, but are not necessarily sequentially performed. For example, the sequential position of each operation may be changed, or at least two operations may be performed in parallel.

According to one or more embodiments, it may be understood that operation 301, operation 302, operation 303, operation 304, operation 305, operation 306, operation 307, operation 308, operation 309, operation 310, operation 311, operation 312, operation 313, operation 314, and operation 315 are performed by a processor (e.g., the processor 120 in FIG. 1) of the electronic device (e.g., the electronic device 101 in FIG. 1).

In one or more embodiments of the disclosure, in operation 301, under control of the processor 120, the electronic device (e.g., the electronic device 101 in FIG. 1) may determine that there is relevance to a specific exercise based on a signal detected by means of an inertia sensor. The inertia sensor may detect inertia forces of motion to detect information related to the acceleration, velocity, direction, and distance of a moving object to be measured. For example, the inertia sensor may correspond to an acceleration sensor and/or a gyro sensor included in the sensor module (e.g., the sensor module 176 in FIG. 1).

In one or more embodiments of the disclosure, in operation 301, under control of the processor 120, the electronic device 101 may determine that there is relevance to a specific exercise in case that a signal detected through the inertia sensor has been detected for a predetermined time period (e.g., 2 minutes) or more.

For example, the signal detected through the inertia sensor may have a specific pattern. In case that a specific pattern persists and/or repeats for a specific time period, in operation 301, under control of the processor 120, the electronic device 101 may determine that it is a specific exercise.

For example, the specific exercise may be a cycling exercise.

In one or more embodiments of the disclosure, in operation 303, under control of the processor 120, the electronic device 101 may determine whether the GNSS sensor is available.

The GNSS sensor may be included in a communication module (e.g., the communication module 190 in FIG. 1). The GNSS sensor may include, for example, a global positioning system (GPS), a global navigation satellite system (GLONASS), Galileo, Beidou, a quasi-zenith satellite system (QZSS), and/or a navigation with Indian constellation (NAVIC).

The electronic device 101 configures the GNSS sensor to be in a standby state to reduce current consumption of the GNSS sensor. However, the electronic device 101 may, in case that a signal pattern associated with the specific exercise is detected through the inertia sensor, activate the GNSS sensor and identify whether it is the specific exercise according to the signal pattern.

In one or more embodiments of the disclosure, in operation 303, under control of the processor 120, the electronic device 101 may determine whether a GNSS signal is detected through the GNSS sensor.

In one or more embodiments of the disclosure, in operation 303, under control of the processor 120, the electronic device 101 may, in case that the GNSS signal is detected through the GNSS sensor, determine that the GNSS sensor is available and proceed to operation 305.

In one or more embodiments of the disclosure, in operation 303, under control of the processor 120, the electronic device 101 may, in case that no GNSS signal is detected through the GNSS sensor, determine that the GNSS sensor is unavailable and proceed to operation 311.

For example, a GNSS signal may be shaded when the electronic device 101 enters a tunnel or an interior of a building (e.g., a parking lot), and the GNSS signal may be difficult to be detected by a GNSS sensor.

In one or more embodiments of the disclosure, the electronic device 101 may, in case that the GNSS signal is detected through the GNSS sensor, determine whether the user is performing a specific exercise, by using the heart rate (HR) sensor, without information for a movement speed.

For example, when the user is performing a specific exercise, the heart rate may be greater than a designated heart rate.

In one or more embodiments of the disclosure, in operation 305, under control of the processor 120, the electronic device 101 may identify a movement speed of the electronic device 101 based on the GNSS sensor.

The electronic device 101 according to one or more embodiments of the disclosure is assumed to be worn or carried by the user. Accordingly, in operation 305, the operation in which the electronic device 101 identifies the movement speed of the electronic device 101 based on the GNSS sensor may correspond to an operation of identifying the movement speed of the user.

In one or more embodiments of the disclosure, in operation 307, under control of the processor 120, the electronic device 101 may determine whether the movement speed identified based on the GNSS sensor is within a first designated speed range. For example, the first designated speed range may be from 7 to 20 km/h.

In one or more embodiments of the disclosure, in operation 307, under control of the processor 120, the electronic device 101 may, in case that the movement speed is within the designated speed range, proceed to operation 315.

In one or more embodiments of the disclosure, in operation 307, under control of the processor 120, the electronic device 101 may, in case that the movement speed is not within the designated speed range, proceed to operation 309.

In one or more embodiments of the disclosure, in operation 309, under control of the processor 120, the electronic device 101 may determine whether the movement speed identified based on the GNSS sensor is within a second designated speed range. For example, the second designated speed range may be from 20.1 to 70 km/h.

In one or more embodiments of the disclosure, in case that the movement speed identified based on the GNSS sensor is not within the first designated speed range and the second designated speed range in operation 307 and operation 309, under control of the processor 120, the electronic device 101 may determine that the user is not performing the specific exercise.

In one or more embodiments of the disclosure, in case that the electronic device 101 is within the second designated speed range (e.g., 20.1 to 70 km/h), the electronic device 101 may measure the heart rate by using a biometric sensor (e.g., the heart rate (HR) sensor) included in the sensor module 176.

In one or more embodiments of the disclosure, in operation 311, under control of the processor 120, the electronic device 101 may identify the heart rate by using the heart rate (HR) sensor.

In one or more embodiments of the disclosure, in operation 313, under control of the processor 120, the electronic device 101 may determine whether the identified heart rate of the user exceeds the designated heart rate.

In one or more embodiments of the disclosure, in operation 313, under control of the processor 120, the electronic device 101 may determine whether the identified heart rate of the user is higher than the designated heart rate. For example, the designated heart rate may be 120 bpm (beats per minute).

In one or more embodiments of the disclosure, in operation 313, under control of the processor 120, the electronic device 101 may, in case that the identified heart rate of the user is less than or equal to the designated heart rate, determine that there is no relevance to the specific exercise.

In one or more embodiments of the disclosure, in case that it is determined that the identified heart rate of the user exceeds the designated heart rate in operation 313, under control of the processor 120, the electronic device 101 may proceed to operation 315.

In one or more embodiments of the disclosure, in case that it is determined that the identified heart rate of the user is higher than the designated heart rate in operation 313, under control of the processor 120, the electronic device 101 may proceed to operation 315.

In one or more embodiments of the disclosure, in operation 315, under control of the processor 120, the electronic device 101 may determine that the user is performing the specific exercise and execute a function. The function executed by the electronic device 101 is an operation of displaying a notification associated with the specific exercise on a user interface on the display module (e.g., the display module 160 in FIG. 1) including the display. The function executed by the electronic device 101 may store, in the memory (e.g., the memory 130 in FIG. 1), an exercise record before the notification is displayed on the user interface on the display module 160 including the display and an exercise record after the notification is displayed.

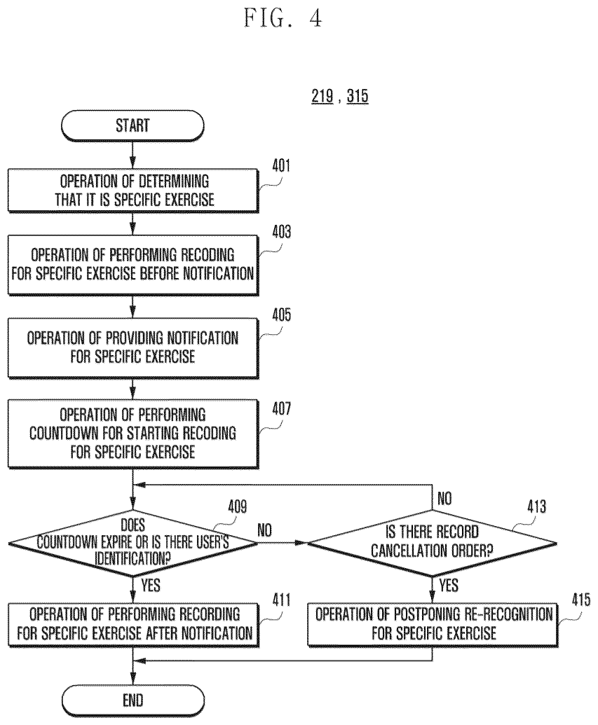

FIG. 4 is a flowchart illustrating operation 219 in FIG. 2 and operation 315 in FIG. 3 according to one or more embodiments of the disclosure.

In the following embodiment, respective operations may be sequentially performed, but are not necessarily sequentially performed. For example, the sequential position of each operation may be changed, or at least two operations may be performed in parallel.

According to one or more embodiments, it may be understood that operation 401, operation 402, operation 403, operation 404, operation 405, operation 406, operation 407, operation 408, operation 409, operation 410, operation 411, operation 412, operation 413, operation 414, and operation 415 are performed by a processor (e.g., the processor 120 in FIG. 1) of the electronic device (e.g., the electronic device 101 in FIG. 1).

In one or more embodiments of the disclosure, in operation 401, under control of the processor 120, the electronic device (e.g., the electronic device 101 in FIG. 1) may determine the specific exercise based on a signal acquired based on the inertia sensor, the GNSS sensor, and/or the HR sensor. For example, the specific exercise may be cycling. The operation of determining the specific exercise based on a signal acquired based on the inertia sensor, the GNSS sensor, and/or the HR sensor has been described with reference to FIG. 2 and FIG. 3.

In one or more embodiments of the disclosure, in operation 403, under control of the processor 120, the electronic device 101 may perform recording for the specific exercise before the notification. The record of the specific exercise before the notification occurs may include information such as the travel time, travel distance, speed, maximum speed, current speed, average speed, and movement location (e.g., latitude and longitude) when the user performs the specific exercise, as well as the maximum heart rate when performing the exercise, average heart rate when performing the exercise, and/or altitude when performing the exercise.

In one or more embodiments of the disclosure, the record of the specific exercise before the notification may correspond to a record acquired through the inertia sensor and the HR sensor before the electronic device 101 determines that the specific exercise is being performed through the GNSS sensor and the HR sensor.

For example, the electronic device 101 may collect records associated with the specific exercise through the inertia sensor and the HR sensor even while determining the specific exercise through the GNSS sensor and the HR sensor from operation 201, operation 202, operation 203, operation 204, operation 205, operation 206, operation 207, operation 208, operation 209, operation 210, operation 211, operation 212, operation 213, operation 214, operation 215, operation 216, operation 217, operation 218, and operation 219 in FIG. 2.

For example, the electronic device 101 may collect records associated with the specific exercise through the inertia sensor and the HR sensor even while determining the specific exercise through the GNSS sensor and the HR sensor from operation 301, operation 302, operation 303, operation 304, operation 305, operation 306, operation 307, operation 308, operation 309, operation 310, operation 311, operation 312, operation 313, operation 314, and operation 315 in FIG. 3.

In one or more embodiments of the disclosure, in operation 403, under control of the processor 120, the operation of the electronic device 101 performing the recording for the specific exercise before the notification may include an operation of weighting the heart rate of the user or the altitude during the movement, based on an average speed acquired using the inertia sensor so as to determine a movement distance.

In one or more embodiments of the disclosure, in operation 405, under control of the processor 120, the electronic device 101 may provide a notification for the specific exercise.

In one or more embodiments of the disclosure, in operation 405, under control of the processor 120, the electronic device 101 may display the notification notifying that the specific exercise is being performed, on the display module (e.g., the display module 160 in FIG. 1) including the display.

In one or more embodiments of the disclosure, in operation 405, under control of the processor 120, the electronic device 101 may provide the notification notifying that the specific exercise is being performed as a sound through a speaker.

In one or more embodiments of the disclosure, in operation 405, under control of the processor 120, the electronic device 101 may provide the notification notifying that the specific exercise is being performed in a tactile manner (e.g., vibration) through a haptic module (e.g., the haptic module 179 in FIG. 1).

In one or more embodiments of the disclosure, in operation 407, under control of the processor 120, the electronic device 101 may perform an operation of counting down for starting recording for the specific exercise.

In one or more embodiments of the disclosure, in operation 407, under control of the processor 120, the electronic device 101 may display the operation of counting down for starting recording for the specific exercise on the display module 160 including the display.

In one or more embodiments of the disclosure, in operation 407, under control of the processor 120, the electronic device 101 may provide the operation of counting down for starting recording for the specific exercise as a sound through the speaker.

In one or more embodiments of the disclosure, in operation 407, under control of the processor 120, the electronic device 101 may provide the operation of counting down for starting recording for the specific exercise in a tactile manner (e.g., vibration) through the haptic module 179.

In one or more embodiments of the disclosure, in operation 409, under control of the processor 120, the electronic device 101 may determine whether the countdown expires or there is identification of the user with respect to the notification.

In case that the countdown expires or there is identification of the user with respect to the notification, the electronic device 101 may proceed from operation 409 to operation 411.

In case that the countdown expires, the electronic device 101 may automatically perform recording for the specific exercise.

In case that the countdown does not expires and there is no identification of the user with respect to the notification, the electronic device 101 may proceed from operation 409 to operation 413.

In one or more embodiments of the disclosure, in operation 413, under control of the processor 120, the electronic device 101 may determine whether there is a record cancellation order. The record cancellation order may correspond to an order to delete a record for a specific exercise and cancel a notification.

In one or more embodiments of the disclosure, in case that there is the record cancellation order, under control of the processor 120, the electronic device 101 may proceed from operation 413 to operation 415.

In one or more embodiments of the disclosure, in operation 415, under control of the processor 120, the electronic device 101 may postpone re-recognition of a specific exercise.

In one or more embodiments of the disclosure, in case that there is no record cancellation order, under control of the processor 120, the electronic device 101 may proceed from operation 413 to operation 409.

In one or more embodiments of the disclosure, in operation 411, under control of the processor 120, the electronic device 101 may perform recording for the specific exercise after the notification.

In one or more embodiments of the disclosure, a record for the specific exercise after the notification may correspond to a record acquired based on the GNSS sensor and the HR sensor. The record of a specific exercise after the notification may be a combined record with the previously acquired record of the specific exercise from operation 403.

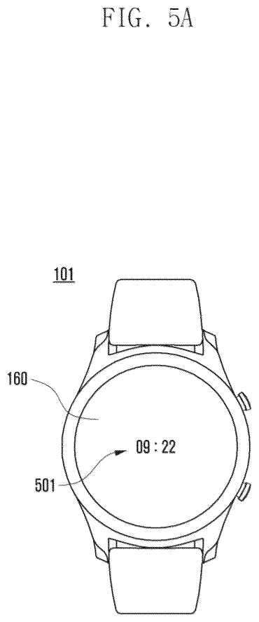

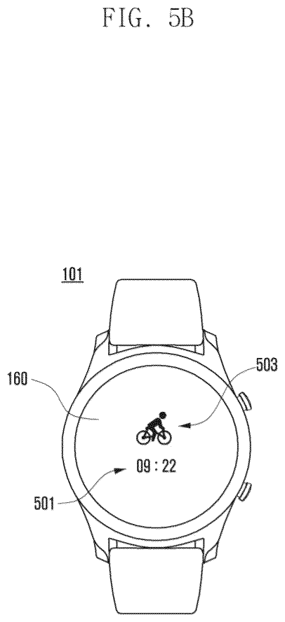

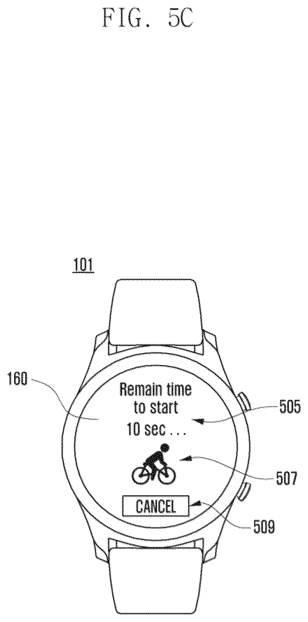

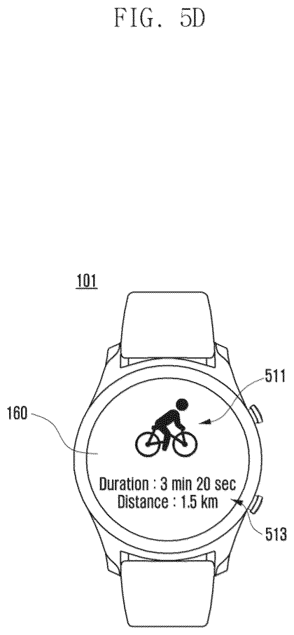

FIG. 5A is a view illustrating a screen before an electronic device 101 identifies a specific exercise according to one or more embodiments of the disclosure.

Before identifying the specific exercise, the electronic device 101 may display a clock screen 501, a lock screen, and/or a standby screen on the display module 160 including the display.

FIG. 5B illustrates a screen while an electronic device 101 recognizes whether it is a specific exercise according to one or more embodiments of the disclosure.

In FIG. 5B, in case that it is determined that there is relevance to a specific exercise based on a signal detected by means of an inertia sensor such as operation 201 in FIG. 2 or operation 301 in FIG. 3, the electronic device 101 may start to determine whether the specific exercise is being performed by using the GNSS sensor and the HR sensor. Here, the electronic device 101 may display a user interface 503 associated with the specific exercise (e.g., cycling), together with the clock screen 501, the lock screen, and/or the standby screen, on the display module 160 including the display. For example, the user interface 503 associated with the specific exercise (e.g., cycling) may correspond to a display object (e.g., an icon) visualizing a motion of the specific exercise.

FIG. 5C illustrates a screen where an electronic device 101 determines that it is a specific exercise and displays a notification according to one or more embodiments of the disclosure.

In case that it is determined by using the inertia sensor, the GNSS sensor, and the HR sensor that the user is performing the specific exercise (e.g., operation 219 in FIG. 2 or operation 315 in FIG. 3), the electronic device 101 may display a user interface for the notification on the display module 160 including the display. For example, the user interface for the notification may include a user interface such as a countdown 505 (e.g., remain time to start 10 sec.), a user interface associated with a specific exercise 507 (e.g., cycling) (e.g., an icon such as cycling), and/or a user interface such as cancel 509 (e.g., cancel).

For example, the cancel 509 may correspond to an order to delete a record for a specific exercise and cancel a notification. When the countdown (e.g., remain time to start 10 sec.) 505 is completed, recording for a specific exercise may automatically start and the user interface for cancel 509 may be removed.

FIG. 5D illustrates a screen where an electronic device 101 performs recording for a user's specific exercise by using a GNSS sensor and an HR sensor according to one or more embodiments of the disclosure.

In FIG. 5D, in case that the countdown expires or the user identifies the notification, the electronic device 101 may automatically perform recording for the specific exercise.

While performing the recording for the specific exercise, the electronic device 101 may display a user interface 511 (e.g., an icon such as cycling) associated with the specific exercise (e.g., cycling) and/or a user interface 513 associated with a movement duration (e.g., duration: 3 min 20 sec) and a movement distance (e.g., distance: 1.5 km) on the display module 160 including the display.

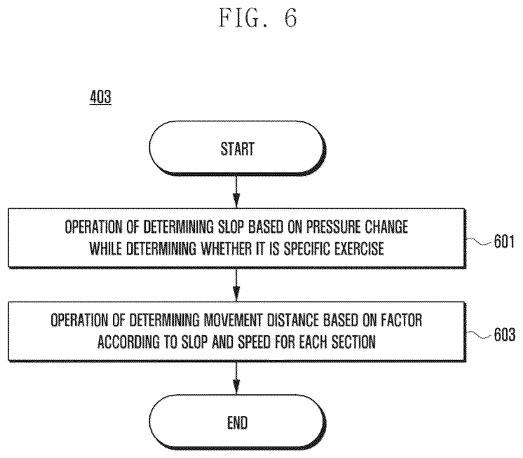

FIG. 6 is a flowchart illustrating a method for determining a movement distance in an operation 403 of performing recording for a specific exercise before a notification in FIG. 4 according to one or more embodiments of the disclosure.

In the following embodiment, respective operations may be sequentially performed, but are not necessarily sequentially performed. For example, the sequential position of each operation may be changed, or at least two operations may be performed in parallel.

According to one or more embodiments, it may be understood that operation 601 and operation 603 are performed by a processor (e.g., the processor 120 in FIG. 1) of the electronic device (e.g., the electronic device 101 in FIG. 1).

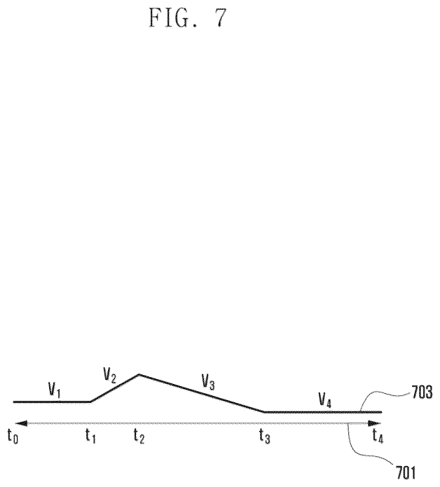

FIG. 7 is a graph illustrating a method for determining a movement distance in FIG. 6 according to one or more embodiments of the disclosure.

In one or more embodiments of the disclosure, in operation 601, under control of the processor 120, the electronic device (e.g., the electronic device 101 in FIG. 1) may determine a slope (or altitude) based on a pressure change while determining whether it is the specific exercise based on the inertia sensor, the GNSS sensor, and/or the HR sensor. The electronic device 101 may detect a pressure change of the electronic device 101 based on a pressure sensor included in the sensor module (e.g., the sensor module 176 in FIG. 1) and determine the slop (or altitude) based on the pressure change.

In one or more embodiments of the disclosure, in operation 603, under control of the processor 120, the electronic device 101 may determine the movement distance based on factors such as a speed and a slope for each segment.

Referring to FIG. 6 and FIG. 7, graph 701 represents time changes and graph 703 represents slopes over time.

A speed during a first time period (t0 to t1) may be a first speed v1, a speed during a second time period (t1 to t2) may be a second speed v2, a speed during a third time period (t2 to t3) may be a third speed v3, and a speed during a fourth time period (t3 to t4) may be a fourth speed v4.

During the first time period (t0 to t1) and the fourth time period (t3 to t4), the electronic device 101 may move over substantially level ground. During the second time period (t1 to t2), the electronic device 101 may move uphill. During the third time period (t2 to t3), the electronic device 101 may move downhill.

If the speed of the electronic device 101 while traveling on substantially level ground has a weight (or factor) of 1, a weight (or factor) may be less than 1 for the speed while the electronic device 101 is moving uphill, and a weight (or factor) may be greater than 1 for the speed while the electronic device 101 is moving downhill. The electronic device 101 may determine a movement distance based on the speed and time according to a weight (or factor).

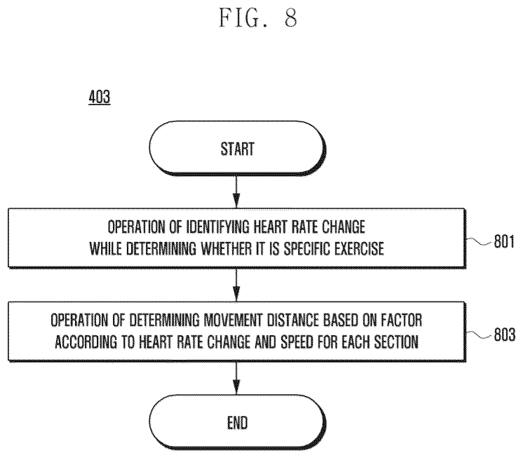

FIG. 8 is a flowchart illustrating a method for determining a movement distance in an operation 403 of performing recording for a specific exercise before a notification in FIG. 4 according to one or more embodiments of the disclosure.

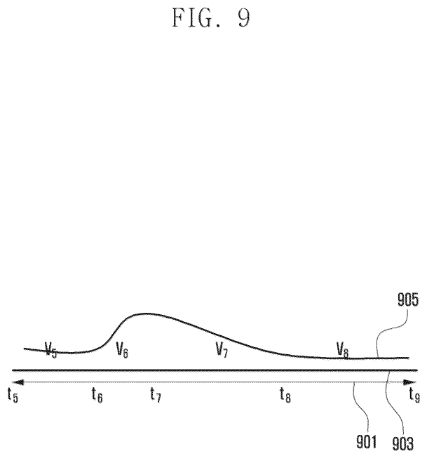

FIG. 9 is a graph illustrating a method for determining a movement distance in FIG. 8 according to one or more embodiments of the disclosure.

In the following embodiment, respective operations may be sequentially performed, but are not necessarily sequentially performed. For example, the sequential position of each operation may be changed, or at least two operations may be performed in parallel.

According to one or more embodiments, it may be understood that operation 801 and operation 803 are performed by a processor (e.g., the processor 120 in FIG. 1) of the electronic device (e.g., the electronic device 101 in FIG. 1).

In one or more embodiments of the disclosure, in operation 801, under control of the processor 120, the electronic device 101 may identify a heart rate change while determining whether it is the specific exercise based on the inertia sensor, the GNSS sensor, and/or the HR sensor.

In one or more embodiments of the disclosure, in operation 803, under control of the processor 120, the electronic device 101 may determine the movement distance based on factors such as a speed and a heart rate change for each segment. For example, at higher speeds, the heart rate may be higher than a determined heart rate. The electronic device 101 may compensate for the speed by weighting heart rate changes.

Referring to FIG. 8 and FIG. 9, graph 901 represents time changes, graph 903 represents slopes over time, and graph 905 represents heart rate changes over time.

A speed during a fifth time period (t5 to t6) may be a fifth speed v5, a speed during a sixth time period (t6 to t7) may be a sixth speed v6, a speed during a seventh time period (t7 to t8) may be a seventh speed v7, and a speed during an eighth time period (t8 to t9) may be an eighth speed v8.

During the fifth time period (T5 to T6), and the eighth time period (t8 to t9), if the speed of the electronic device 101 during substantially no heart rate change has a weight (or factor) of 1, the weight (or factor) of the speed during an increase in heart rate change may be greater than 1, and the weight (or factor) of the speed during a decrease in heart rate change may be less than 1. The electronic device 101 may determine a movement distance based on the speed and time according to a weight (or factor).

In one or more embodiments, an electronic device 101 may include an inertia sensor, a global navigation satellite system (GNSS) sensor, a heat rate (HR) sensor, a memory 130, a display module 160 including a display, and a processor 120.

In one or more embodiments, the processor 120 may determine that there is relevance to a specific exercise, based on a signal detected by means of the inertia sensor, determine whether the GNSS sensor is available, identify the movement speed of the electronic device 101 based on the GNSS sensor, in case that the identified movement speed is greater than or equal to a first specific speed, determine whether the identified movement speed is greater than a second specific speed, in case that the identified movement speed is less than the second specific speed, determine whether the identified movement speed is within a designated speed range, and in case that the identified movement speed is within the designated speed range, determine that a user is performing the specific exercise and execute a function.

In one or more embodiments, the processor 120 may, in case that the identified movement speed is not within the designated speed range, identify a heart rate of the user by using the HR sensor, and, in case that the identified heart rate exceeds a specific heart rate and exceeds a designated heart rate range, determine that the user is performing the specific exercise and execute a function.

In one or more embodiments, the processor 120 may, in case that the identified heart rate exceeds the specific heart rate and is within the designated heart rate range, monitor the heart rate of the user by using the HR sensor.

In one or more embodiments, the designated speed range may be greater than the first specific speed and less than the second specific speed.

In one or more embodiments, the processor 120 may, in case that the GNSS sensor is not available, identify the heart rate of the user by using the HR sensor, and, in case that the identified heart rate exceeds a specific heart rate and exceeds a designated heart rate range, determine that the user is performing the specific exercise and execute a function.

In one or more embodiments, the processor 120 may determine the specific exercise based on a signal acquired based on the inertia sensor, the GNSS sensor, and/or the HR sensor, perform recording with respect to the specific exercise before the inertia sensor and an inertia sensor notification, display a notification for the specific exercise on the display module 160, and perform a countdown for starting recording for the specific exercise.

In one or more embodiments, the processor 120 may, in case that the countdown is expired or there is a user's identification, perform recording for the specific exercise after the notification.

In one or more embodiments, the processor 120 may aggregate and store a record for the specific exercise before the notification and a record for the specific exercise after the notification and display the stored record.

In one or more embodiments, the processor 120 may determine a slope based on a pressure change while determining whether it is the specific exercise and determine a movement distance based on a factor according to the slope and a speed for each section.

In one or more embodiments, the processor 120 may determine a heart rate change of the user while determining whether it is the specific exercise and determine the movement distance based on a factor according to the heart rate change of the user and the speed for each section.

In one or more embodiments, a method for detecting an exercise of a user of an electronic device 101 may include an operation of determining that there is relevance to a specific exercise based on a signal detected by means of the inertia sensor, an operation of determining whether the GNSS sensor is available, an operation of identifying the movement speed of the electronic device 101 based on the GNSS sensor, an operation of, in case that the identified movement speed is greater than or equal to a first specific speed, determining whether the identified movement speed is greater than a second specific speed, an operation of, in case that the identified movement speed is less than the second specific speed, determining whether the identified movement speed is within a designated speed range, and an operation of, in case that the identified movement speed is within the designated speed range, determining that a user is performing the specific exercise and executing a function.

In one or more embodiments, the method for detecting an exercise of the user of the electronic device 101 may include an operation of, in case that the identified movement speed is not within the designated speed range, identifying a heart rate of the user by using the

HR sensor, and an operation of, in case that the identified heart rate exceeds a specific heart rate and exceeds a designated heart rate range, determining that the user is performing the specific exercise and executing a function.

In one or more embodiments, the method for detecting an exercise of the user of the electronic device 101 may include an operation of, in case that the identified heart rate exceeds the specific heart rate and is within the designated heart rate range, monitoring the heart rate of the user by using the HR sensor.

In one or more embodiments, the method for detecting an exercise of the user of the electronic device 101 may include an operation of, in case that the GNSS sensor is not available, identifying the heart rate of the user by using the HR sensor, and an operation of, in case that the identified heart rate exceeds a specific heart rate and exceeds a designated heart rate range, determining that the user is performing the specific exercise and executing a function.

In one or more embodiments, the method for detecting an exercise of the user of the electronic device 101 may include an operation of determining the specific exercise based on a signal acquired based on the inertia sensor, the GNSS sensor, and/or the HR sensor, an operation of performing recording with respect to the specific exercise before the inertia sensor and an inertia sensor notification, and an operation of displaying a notification for the specific exercise on the display module 160, and performing a countdown for starting recording for the specific exercise.

In one or more embodiments, the method for detecting an exercise of the user of the electronic device 101 may include an operation of, in case that the countdown is expired or there is a user's identification, performing recording for the specific exercise after the notification.

In one or more embodiments, the method for detecting an exercise of the user of the electronic device 101 may include an operation of aggregating and storing a record for the specific exercise before the notification and a record for the specific exercise after the notification and displaying the stored record.

In one or more embodiments, the method for detecting an exercise of the user of the electronic device 101 may include an operation of determining a slope based on a pressure change while determining whether it is the specific exercise and determining a movement distance based on a factor according to the slope and a speed for each section.

In one or more embodiments, the method for detecting an exercise of the user of the electronic device 101 may include an operation of determining a heart rate change of the user while determining whether it is the specific exercise and determining the movement distance based on a factor according to the heart rate change of the user and the speed for each section.

The electronic device according to various embodiments set forth herein may be one of various types of electronic devices. The electronic device may include, for example, a portable communication device (e.g., a smart phone), a computer device, a portable multimedia device, a portable medical device, a camera, a wearable device, or a home appliance. The electronic device according to embodiments of the disclosure is not limited to those described above.

It should be appreciated that various embodiments of the disclosure and the terms used therein are not intended to limit the technological features set forth herein to particular embodiments and the disclosure includes various changes, equivalents, or alternatives for a corresponding embodiment. With regard to the description of the drawings, similar reference numerals may be used to designate similar or relevant elements. A singular form of a noun corresponding to an item may include one or more of the items, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C” may include all possible combinations of the items enumerated together in a corresponding one of the phrases. Such terms as “a first,” “a second,” “the first,” and “the second” may be used to simply distinguish a corresponding element from another, and does not limit the elements in other aspect (e.g., importance or order). If an element (e.g., a first element) is referred to, with or without the term “operatively” or “communicatively”, as “coupled with/to” or “connected with/to” another element (e.g., a second element), it means that the element may be coupled/connected with/to the other element directly (e.g., wiredly), wirelessly, or via a third element.

As used herein, the term “module” may include a unit implemented in hardware, software, or firmware, and may interchangeably be used with other terms, for example, “logic”, “logic block”, “part”, or “circuitry”. The “module” may be a single integrated component, or a minimum unit or part thereof, adapted to perform one or more functions. For example, according to one or more embodiments, the “module” may be implemented in the form of an application-specific integrated circuit (ASIC).

Various embodiments as set forth herein may be implemented as software (e.g., the program 140) including one or more instructions that are stored in a storage medium (e.g., the internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Herein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to one or more embodiments, methods according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each element (e.g., a module or a program) of the above-described elements may include a single entity or multiple entities. According to various embodiments, one or more of the above-described elements or operations may be omitted, or one or more other elements or operations may be added. Alternatively or additionally, a plurality of elements (e.g., modules or programs) may be integrated into a single element. In such a case, according to various embodiments, the integrated element may still perform one or more functions of each of the plurality of elements in the same or similar manner as they are performed by a corresponding one of the plurality of elements before the integration. According to various embodiments, operations performed by the module, the program, or another clement may be carried out sequentially, in parallel, repeatedly, or heuristically, or one or more of the operations may be executed in a different order or omitted, or one or more other operations may be added.

What is claimed is:

1. An electronic device comprising:
an inertia sensor;
a global navigation satellite system (GNSS) sensor;
a heart rate (HR) sensor;
memory, including one or more storage media, storing instructions;
a display; and
at least one processor including processing circuitry,
wherein the instructions, when executed by the at least one processor individually or collectively, cause the electronic device to:
identify a specific exercise by differentiating the specific exercise from other exercises, based on a signal detected through the inertia sensor;
determine whether the GNSS sensor is available;
identify a movement speed of the electronic device based on the GNSS sensor;
based on determining that the identified movement speed is greater than or equal to a first specific speed, determine whether the identified movement speed is greater than a second specific speed;
based on determining that the identified movement speed is less than the second specific speed, determine whether the identified movement speed is within a designated speed range; and
based on determining that the identified movement speed is within the designated speed range, determine that a user is performing the specific exercise and execute a function of the electronic device.

2. The electronic device of claim 1, wherein the instructions, when executed by the at least one processor individually or collectively, cause the electronic device to:
based on determining that the identified movement speed is not within the designated speed range, identify a heart rate of the user by using the HR sensor; and
based on determining that the identified heart rate exceeds a specific heart rate and exceeds a designated heart rate range, determine that the user is performing the specific exercise and execute the function.

3. The electronic device of claim 2, wherein the instructions, when executed by the at least one processor individually or collectively, cause the electronic device to:
based on determining that the identified heart rate exceeds the specific heart rate and is within the designated heart rate range, monitor the heart rate of the user by using the HR sensor.

4. The electronic device of claim 1, wherein the designated speed range is greater than the first specific speed and is less than the second specific speed.

5. The electronic device of claim 1, wherein the instructions, when executed by the at least one processor individually or collectively, cause the electronic device to:
based on determining that the GNSS sensor is not available, identify the heart rate of the user by using the HR sensor; and
based on determining that the identified heart rate exceeds a specific heart rate and exceeds the designated heart rate range, determine that the user is performing the specific exercise and execute the function.

6. The electronic device of claim 1, wherein the instructions, when executed by the at least one processor individually or collectively, cause the electronic device to:
determine the specific exercise based on a signal acquired based on at least one of the inertia sensor, the GNSS sensor, and the HR sensor;
perform a recording of the inertia sensor and the specific exercise before a notification of the inertia sensor; and
display a notification for the specific exercise on the display, and perform a countdown for starting recording for the specific exercise.

7. The electronic device of claim 6, wherein the instructions, when executed by the at least one processor individually or collectively, cause the electronic device to:
based on determining that the countdown is expired or a user's identification being identified, perform recording of the specific exercise after the notification.

8. The electronic device of claim 6, wherein the instructions, when executed by the at least one processor individually or collectively, cause the electronic device to:
aggregate and store a first record of the specific exercise before the notification is displayed and a second record for the specific exercise after the notification, and display an aggregated record of an aggregation of the stored first record and the stored second record.

9. The electronic device of claim 6, wherein the instructions, when executed by the at least one processor individually or collectively, cause the electronic device to:
determine a slope based on a pressure change while determining whether the user is performing the specific exercise.

10. The electronic device of claim 6, wherein the instructions, when executed by the at least one processor individually or collectively, cause the electronic device to:
determine a heart rate change of the user while determining whether the user is performing the specific exercise; and
determine the movement distance based on a factor according to the heart rate change of the user and the speed for each of the plurality of sections.

11. A method of detecting an exercise of a user of an electronic device, the method comprising:
identifying a specific exercise by differentiating the specific exercise from other exercises, based on a signal detected through an inertia sensor;
determining whether a global navigation satellite system (GNSS) sensor of the electronic device is available;
identifying a movement speed of the electronic device, based on the GNSS sensor;
based on determining that the identified movement speed is greater than or equal to a first specific speed, determining whether the identified movement speed is greater than a second specific speed;
based on determining that the identified movement speed is less than the second specific speed, determining whether the identified movement speed is within a designated speed range; and
based on determining that the identified movement speed is within the designated speed range, determining that the user is performing the specific exercise and executing a function of the electronic device.

12. The method of claim 11, further comprising:
based on determining that the identified movement speed is not within the designated speed range, identifying a heart rate of the user by using a heart rate (HR) sensor; and
based on determining that the identified heart rate exceeds a specific heart rate and exceeds a designated heart rate range, determining that the user is performing the specific exercise and executing a function.

13. The method of claim 12, further comprising:
based on determining that the identified heart rate exceeds the specific heart rate and is within the designated heart rate range, monitoring the heart rate of the user by using the HR sensor.

14. The method of claim 11, wherein the designated speed range is greater than the first specific speed and is less than the second specific speed.

15. The method of claim 11, further comprising:
based on determining that the GNSS sensor is not available, identifying the heart rate of the user by using the HR sensor; and
based on determining that the identified heart rate exceeds a specific heart rate and exceeds the designated heart rate range, determining that the user is performing the specific exercise and executing the function.

16. The method of claim 11, further comprising:
determining the specific exercise based on a signal acquired based on at least one of the inertia sensor, the GNSS sensor, and the HR sensor;
performing a recording of the inertia sensor and the specific exercise before a notification of the inertia sensor; and
displaying a notification for the specific exercise on a display of the electronic device, and performing a countdown for starting recording for the specific exercise.

17. The method of claim 16, further comprising:
based on determining that the countdown is expired or that a user's identification being identified, performing recording of the specific exercise after the notification.

18. The method of claim 17, further comprising:
aggregating and storing a first record of the specific exercise before the notification is displayed and a second record for the specific exercise after the notification and displaying an aggregated record of an aggregation of the stored first record and the stored second record.

19. The method of claim 16, further comprising:
determining a slope based on a pressure change while determining whether the user is performing the specific exercise; and
determining a movement distance based on a factor according to the slope and a speed for each of a plurality of sections from the recording.

20. The method of claim 16, further comprising:
determining a heart rate change of the user while determining whether the user is performing the specific exercise; and
determining a movement distance based on a factor according to the heart rate change of the user and the speed for each of a plurality of sections of the recording.
